(19) United States
(12) Patent Application Publication
(10) Pub. No.: US 2026/0226131 A1
(43) Pub. Date: Aug. 6, 2026

(54) LONG-ACTING INSULIN-FC FUSION PROTEIN

(71) Applicant: SHANGHAI INSTITUTE OF MATERIA MEDICA, CHINESE ACADEMY OF SCIENCES, Shanghai (CN)

(72) Inventors: Chunhe WANG, Shanghai (CN); Jia LI, Shanghai (CN); Yili SUN, Shanghai (CN); Min PEI, Shanghai (CN); Yili CHEN, Shanghai (CN); Chunmei XIA, Shanghai (CN); Guifeng WANG, Shanghai (CN); Min WANG, Shanghai (CN); QI WANG, Shanghai (CN); Tiancheng DONG, Shanghai (CN); Chaoying HUANG, Shanghai (CN)

(21) Appl. No.: 19/148,462

(22) PCT Filed: Dec. 8, 2023
(86) PCT No.: PCT/CN2023/137326

§ 371 Date: Jul. 16, 2025

(30) Foreign Application Priority Data
Jan. 16, 2023 (CN) 202310075021.9

(51) Int. Cl. C07K 14/62; A61K 38/00; A61P 3/10

(52) U.S. Cl. CPC C07K 14/62; A61P 3/10; A61K 38/00; C07K 2319/30

(57) ABSTRACT

The present invention provides a long-acting insulin-Fc fusion protein. The insulin-Fc fusion protein of the present invention has reduced insulin receptor affinity, improved selectivity to auxin receptors, and a prolonged half-life in vivo, and can prolong a glucose lowering duration in vivo, reduce the time interval of insulin usage, and reduce the side effects such as a hypoglycemia risk.

### TECHNICAL FIELD

The present invention relates to a fusion protein for the treatment of diabetes. More specifically, the present invention relates to a fusion protein in which an insulin analog is fused to a human IgG Fc region through a peptide linker, a preparation method thereof and use thereof in the treatment of diabetes.

### BACKGROUND ART

Diabetes is a chronic disease characterized by hyperglycemia due to insufficient insulin secretion or/and insulin resistance. Type 1 diabetes is mainly caused by insufficient insulin secretion caused by destruction of pancreatic β cells, and can only be treated with insulin. Type 2 diabetes is mainly caused by insulin resistance and insufficient compensatory secretion of insulin, accounting for about 90-95% in all cases, and insulin therapy is still required when the disease progresses to a later stage.

Exogenous insulin supplementation is one of the important treatment means to improve the life quality of diabetic patients and delay the progression of the disease course. Insulin has a short half-life in the blood, and current conventional therapies are large-dose insulin injection at mealtime, and oral or injection administration of exogenous insulin once or more times per day.

Frequent insulin injections and self-glucose management testing bring many inconveniences and pain to diabetic patients with poor treatment compliance, and currently available products are prone to hypoglycemia and weight gain, so many patients are unwilling to start insulin treatment resulting in poor overall blood glucose control. Even patients who have received insulin treatment cannot strictly follow the doctor's orders to inject insulin to strictly control blood glucose. Poor blood glucose control may be prone to various serious complications, including atherosclerotic cardiovascular diseases, diabetes-related kidney and liver diseases, cancer, infection, etc. So far, no basal insulin drugs with a lower frequency of injection than once a day have been approved, so there is a need for an insulin product with a longer duration of glucose-lowering effect and a lower injection frequency than current products to improve patient treatment compliance.

Currently, basal insulin-Fc fusion proteins with extended half-life have been reported. CN103509118 discloses a protein in which human insulin B chain and human insulin A chain are connected through a 4 to 50 amino acid C-peptide linker sequence and the insulin A chain is directly connected to the immunoglobulin Fc fragment, wherein the protein showed an in vivo half-life of about 3 days in mouse experiments. KR1020150087130 discloses a protein in which a proinsulin analog is connected to an immunoglobulin Fc region by using a non-peptide linker, wherein the protein showed an extended serum half-life over the existing therapies. US20180177851 discloses a protein in which an insulin receptor agonist with reduced insulin receptor affinity is connected to a human Fc region by using a second peptide linker, and a human insulin B chain and a human insulin A chain are connected by a first peptide linker of 5 to 10 amino acids, and this once-weekly insulin fusion protein injection LY3209590 showed similar efficacy and safety compared to daily basal insulin in a phase 2 clinical trial (Frias J P, et al. J Endocr Soc. 2021; 5 (Suppl 1): A448-A449.).

Despite the foregoing and/or other disclosures, there is still a continuing need in the art for insulin products having a prolonged duration of hypoglycemic action.

### SUMMARY OF THE INVENTION

The present invention provides a fusion protein comprising an insulin analog, a peptide linker between the human insulin B chain and the human insulin A chain, and a human IgG Fc fragment to meet clinical needs, achieve a lower frequency of injections, and improve patient compliance.

In order to prolong the action time of insulin, the following design strategies are adopted in the present invention: first, the affinity of the insulin analogue to the insulin receptor is reduced by means of protein engineering, so that the insulin clearance mediated by the insulin receptor is reduced, but sufficient biological efficacy is still retained to activate the receptor and the downstream signaling pathway; second, in order to evade kidney clearance, the insulin analogue is fused to the human immunoglobulin Fc region to increase the fluid dynamic size, but the loss of physiological activity due to insulin self-aggregation caused by dimerization of the Fc fragment is still to be avoided. Therefore, an Fc region needs to be engineered to reduce additional insulin aggregation.

The fusion protein provided by the present invention has a prolonged duration of hypoglycemic effect and a lower frequency of injections compared to the existing once-daily insulin therapy.

The fusion protein provided by the present invention has a flat pharmacokinetic profile and can provide a lower incidence of hypoglycemia.

In an aspect, the present invention provides a fusion protein comprising:
a) an insulin analog having a structure represented by the formula of Z₁-Z₂-Z₃, wherein:
   Z₁ is an insulin B chain analog comprising the amino acid sequence of:

(SEQ ID NO: 1)
FVNQX₁LCGSHLVEALX₂LVCGERGFX₃YX₄X₅X₆X₇

wherein
X₁ is H or A,
X₂ is Y, A or E,
X₃ is T, I, For H,
X₄ is E, D, T or absent,
X₅ is P, E or absent,
X₆ is K, E or absent,
X₇ is T or absent;
Z₂ is a first peptide linker, preferably comprising an amino acid sequence selected from the group consisting of GGVGGG (SEQ ID NO: 16), GGGSGG (SEQ ID NO: 17) and GGGGGV (SEQ ID NO: 18));
Z₃ is an insulin A chain analog comprising the amino acid sequence of:

(SEQ ID NO: 2)
GIX₈EX₉CCX₁₀SICSLYQLENYCX₁₁

wherein
X₈ is A, L or V;
X₉ is Q or A;
X₁₀ is T, A, F or K;
X₁₁ is G, S or N;
b) a second peptide linker; and
c) a human IgG Fc region or a mutant thereof;
wherein the C-terminal residue of the insulin analog is directly fused to the N-terminal residue of the second peptide linker, and the C-terminal residue of the second peptide linker is directly fused to the N-terminal residue of the human IgG Fc region or a mutant thereof.

In an embodiment, the insulin B chain analog comprises a modification to the amino acid sequence of the B chain of the human insulin molecule at X₁ of SEQ ID NO: 1.

In an embodiment, in Z₁, X₁ is A; X₂ is Y; X₃ is F; X₄ is T; X₅ is P; X₆ is K; and X₇ is T.

In an embodiment, the insulin B chain analog comprises a modification to the amino acid sequence of the B chain of the human insulin molecule at X₄.

In an embodiment, in Z₁, X₁ is H; X₂ is A; X₃ is F; X₄ is T; X₅ is P; X₆ is K; and X₇ is T.

In an embodiment, the insulin B chain analog comprises a modification to the amino acid sequence of the B chain of the human insulin molecule at X₅ and X₆.

In an embodiment, in Z₁, X₁ is H; X₂ is Y; X₃ is T or I; X₄ is E; X₅ is P; X₆ is K; and X₇ is T.

In an embodiment, the insulin B chain analog comprises a modification to the amino acid sequence of the B chain of the human insulin molecule at X₆ of SEQ ID NO: 1.

In an embodiment, in Z₁, X₁ is H; X₂ is Y; X₃ is F; X₄ is E or D; X₅ is P; X₆ is K; and X₇ is T.

In an embodiment, the insulin B chain analog comprises a modification to the amino acid sequence of the B chain of the human insulin molecule at X₄, X₅, X₆, and X₇.

In an embodiment, in Z₁, X₄ to X₇ are absent.

In an embodiment, the insulin A chain analog comprises at least one modification to the amino acid sequence of the human insulin A chain at X₈ or X₁₁ of SEQ ID NO: 2.

In an embodiment, the insulin A chain analog comprises at least one modification to the amino acid sequence of the A chain of the human insulin molecule at X₈ or X₉ and X₁₁ of SEQ ID NO: 2.

In an embodiment, the insulin A chain analog comprises a modification to the amino acid sequence of the A chain of the human insulin molecule at X₈ and X₉ and X₁₁ of SEQ ID NO: 2.

In an embodiment, in Z₃, X₈ is A or L; X₉ is A; X₁₀ is T; and X₁₁ is G.

In an embodiment, the insulin A chain analog comprises a modification to the amino acid sequence of the A chain of the human insulin molecule at X₁₁ of SEQ ID NO: 2.

In an embodiment, in Z₃, X₈ is V; X₉ is Q; X₁₀ is T; and X₁₁ is G.

In an embodiment, the insulin A chain analog comprises a modification to the amino acid sequence of the A chain of the human insulin molecule at X₁₀ and X₁₁.

In an embodiment, in Z₃, X₈ is V; X₉ is Q; X₁₀ is A, K, or F; and X₁₁ is G.

In an embodiment, the insulin A chain analog comprises a modification to the amino acid sequence of the A chain of the human insulin molecule at X₈ and X₁₀ and X₁₁.

In an embodiment, in Z₃, X₈ is A or L; X₉ is Q; X₁₀ is A or K; and X₁₁ is G.

In an embodiment, the insulin B chain analog comprises a modification to the amino acid sequence of the B chain of the human insulin molecule at X₆ of SEQ ID NO: 1; and the insulin A chain analog comprises a modification to the amino acid sequence of the A chain of the human insulin molecule at both X₈ and X₁₁.

In an embodiment, in Z₁, X₁ is H; X₂ is Y; X₃ is F; X₄ is E or D; X₅ is P; X₆ is K; X₇ is T; and the insulin A chain analogue comprises a modification to the amino acid sequence of the A chain of the human insulin molecule at both X₈ and X₁₁ of SEQ ID NO: 2, and in Z₃, X₈ is A; and X₁₁ is G.

In an embodiment, the insulin B chain analog comprises a B chain amino acid sequence of a human insulin molecule; and the insulin A chain analog comprises a modification to the amino acid sequence of the A chain of the human insulin molecule at both X₈ and X₁₁ of SEQ ID NO: 2.

In an embodiment, in Z₁, X₁ is H; X₂ is Y; X₃ is F; X₄ is T; X₅ is P; X₆ is K; X₇ is T; and the insulin A chain analog comprises a modification to the amino acid sequence of the A chain of the human insulin molecule at X₈ and X₁₁; wherein X₈ is A or L; and X₁₁ is G.

In an embodiment, the insulin B chain analog comprises the amino acid sequence of the B chain of the human insulin molecule; and the insulin A chain analog comprises a modification to the amino acid sequence of the A chain of the human insulin molecule at both X₉ and X₁₁.

In an embodiment, in Z₁, X₁ is H; X₂ is Y; X₃ is F; X₄ is T; X₅ is P; X₆ is K; X₇ is T; and the insulin A chain analog comprises a modification to the amino acid sequence of the A chain of the human insulin molecule at X₉ and X₁₁, wherein X₉ is A; and X₁₁ is G.

In an embodiment, the insulin B chain analog comprises the amino acid sequence of the B chain of the human insulin molecule; and the insulin A chain analog comprises a modification to the amino acid sequence of the A chain of the human insulin molecule at both X₁₀ and X₁₁.

In an embodiment, in Z₁, X₁ is H; X₂ is Y; X₃ is F; X₄ is T; X₅ is P; X₆ is K; X₇ is T; and the insulin A chain analog comprises a modifications of the amino acid sequence of the A chain of the human insulin molecule at X₁₀ and X₁₁, wherein X₁₀ is A, K or F; and X₁₁ is G.

In an embodiment, the insulin B chain analog comprises a modification to the amino acid sequence of the B chain of the human insulin molecule at X₁; and the insulin A chain analog comprises a modification to the amino acid sequence of the A chain of the human insulin molecule at X₁₁.

In an embodiment, in Z₁, X₁ is A; X₂ is Y; X₃ is F; X₄ is T; X₅ is P; X₆ is K; X₇ is T; and the insulin A chain analog comprises a modification to the amino acid sequence of the A chain of the human insulin molecule at X₁₁.

In an embodiment, the insulin B chain analog comprises a modification to the amino acid sequence of the B chain of the human insulin molecule at X₂; and the insulin A chain analog comprises a modification to the amino acid sequence of the A chain of the human insulin molecule at both X₈ and X₁₁.

In an embodiment, in Z₁, X₁ is H; X₂ is A; X₃ is F; X₄ is T; X₅ is P; X₆ is K; X₇ is T; and the insulin A chain analog comprises a modification to the amino acid sequence of the A chain of the human insulin molecule at both X₈ and X₁₁, wherein X₈ is A; and X₁₁ is G.

In an embodiment, the insulin B chain analog comprises a modification to the amino acid sequence of the B chain of the human insulin molecule at X₃ and X₄ of SEQ ID NO: 1; and the insulin A chain analog comprises a modification to the amino acid sequence of the A chain of the human insulin molecule at both X₈ and X₁₁.

In an embodiment, in Z₁, X₁ is H; X₂ is Y; X₃ is T or I; X₄ is E; X₅ is P; X₆ is K; X₇ is T; and the insulin A chain analog comprises a modification to the amino acid sequence of the A chain of the human insulin molecule at both X₈ and X₁₁.

In an embodiment, the insulin B chain analog comprises a modification to the amino acid sequence of the B chain of the human insulin molecule at X₄, X₅, X₆, and X₇; and the insulin A chain analog comprises a modification to the amino acid sequence of the A chain of the human insulin molecule at X₈ and X₁₁.

In an embodiment, in Z₁, X₄ to X₇ are absent; and the insulin A chain analog comprises a modification to the amino acid sequence of the A chain of the human insulin molecule at X₈ and X₁₁, wherein X₈ is A; and X₁₁ is G.

In an embodiment, the first peptide linker Z₂ comprises an amino acid sequence selected from the group consisting of GGVGGG (SEQ ID NO: 16), GGGSGG (SEQ ID NO: 17), and GGGGGV (SEQ ID NO: 18)).

In an embodiment, the first peptide linker Z₂ comprises the amino acid sequence of: GGVGGG (SEQ ID NO: 16).

In an embodiment, Z₁-Z₂-Z₃ in the above fusion protein as an insulin receptor agonist comprises the amino acid sequence of:

(SEQ ID NO: 19)
FVNQX₁LCGSHLVEALX₂LVCGERGFX₃YX₄X₅X₆X₇GGVGGGGI
X₈EX₉CCX₁₀SICSLYQLENYCX₁₁

wherein X₁ is H or A;
X₂ is Y, A or E;
X₃ is T, I, F or H;
X₄ is E, D, T or absent;
X₅ is P, E or absent;
X₆ is K, E or absent;
X₇ is T or absent;
X₈ is A, L or V;
X₉ is Q or A;
X₁₀ is T, A, F or K;
X₁₁ is G, S or N.

In an embodiment, Z₁-Z₂-Z₃ in the above fusion protein as an insulin receptor agonist has the amino acid sequence of:

(SEQ ID NO: 3)
FVNQHLCGSHLVEALYLVCGERGFFYEPKTGGVGGGGIAEQCCTS
ICSLYQLENYCG

In an embodiment, the second peptide linker comprises a peptide having the sequence of [GGGGX]n, wherein X is S or T; and n is 2, 3, 4, or 5.

In another embodiment, the second peptide linker comprises the amino acid sequence of:

(SEQ ID NO: 20)
GGGGX₁₂GGGGX₁₃GGGGX₁₄GGGGX₁₅

where X₁₂ is S or T; X₁₃ is S or T; X₁₄ is S or T; and X₁₅ is S or T.

In another embodiment, the second peptide linker has the amino acid sequence of:

(SEQ ID NO: 4)
GGGGSGGGGSGGGGSGGGGS

In an embodiment, the human IgG Fc region is an Fc region from a IgG2 or IgG4 antibody.

In an embodiment, the human IgG Fc region is an Fc region from an IgG2 antibody and comprises the amino acid sequence of:

(SEQ ID NO: 5)
ERKX₁₆X₁₇VEX₁₈PPX₁₉PAPPVAGPSVFLFPPKPKDTLMISRT
PEVTCVVVDVSX₂₀EDPEVQFNWYVDGVEVHNAKTKPREEQFNST
FRVVSVLTVX₂₁HQDWLNGKEYKCKVSNKGLPX₂₂X₂₃IEKTISK
TKGQPREPQVYTLPPSREEMTKNQVSLTCLVKGFYPSDIAVEWES
NGQPENNYKTTPPMLDSDGSFFLYSKLTVDKSRWQQGNVFSCSVM
HEALHNHYTQKSLSLSX₂₄GX₂₅

wherein X₁₆ is S or C;
X₁₇ is S or C;
X₁₈ is S or C;
X₁₉ is S or C;
X₂₀ is Q or H;
X₂₁ is L or V;
X₂₂ is S or A;
X₂₃ is S or P;
X₂₄ is L or P;
X₂₅ is K or absent.

In an embodiment, the human IgG Fc region is an Fc region from an IgG4 antibody and comprises the amino acid sequence of:

(SEQ ID NO: 6)
ESKYGPPSPPSPAPEAAGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLG

In an embodiment, the fusion protein of the present invention comprises the amino acid sequence of:

(SEQ ID NO: 7)
FVNQX₁LCGSHLVEALX₂LVCGERGFX₃YX₄X₅X₆X₇GGVGGGGI
X&EX₉CCX₁₀SICSLYQLENYCX₁₁GGGGX₁₂GGGGX₁₃GGGGX₁
4GGGGX₁₅ERKX₁₆X₁₇VEX₁₈PPX₁₉PAPPVAGPSVFLFPPKPK
DTLMISRTPEVTCVVVDVSX₂₀EDPEVQFNWYVDGVEVHNAKTKP
REEQFNSTFRVVSVLTVX₂₁HQDWLNGKEYKCKVSNKGLPX₂₂X₂
3IEKTISKTKGQPREPQVYTLPPSREEMTKNQVSLTCLVKGFYPS
DIAVEWESNGQPENNYKTTPPMLDSDGSFFLYSKLTVDKSRWQQG
NVFSCSVMHEALHNHYTQKSLSLSX₂₄GX₂₅

wherein X₁ is H or A;
X₂ is Y, A or E;
X₃ is T, I, F or H;
X₄ is E, D, T or absent;
X₅ is P, E or absent;
X₆ is K, E or absent;
X₇ is T or absent;
X₈ is A, L or V;
X₉ is Q or A;
X₁₀ is T, A, F or K;
X₁₁ is G, S or N;
X₁₂ is S or T;
X₁₃ is S or T;
X₁₄ is S or T;
X₁₅ is S or T;
X₁₆ is S or C;
X₁₇ is S or C;
X₁₈ is S or C;
X₁₉ is S or C;
X₂₀ is Q or H;
X₂₁ is L or V;
X₂₂ is S or A;
X₂₃ is S or P;
X₂₄ is L or P;
X₂₅ is K or absent.

In an embodiment, the IgG Fc region comprises the amino acid sequence of SEQ ID NO: 7 and further comprises some or all of the amino acids found in the wild-type IgG2 Fc sequence on the N-terminal side of the E residue at position 1 in SEQID NO: 7. In a preferred embodiment, the human IgG Fc region comprises the amino acid sequence of SEQ ID NO: 7, wherein X₁₆ is S; X₁₇ is S; X₁₈ is S; X₁₉ is S; X₂₀ is Q; X₂₁ is L; X₂₂ is S; X₂₃ is S; X₂₄ is L; X₂₅ is absent.

In an embodiment, the fusion protein of the present invention comprises an amino acid sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15. In an embodiment, the fusion protein of the present invention comprises the amino acid sequence of:

(SEQ ID NO: 8)
FVNQHLCGSHLVEALYLVCGERGFFYEPKTGGVGGGGIAEQCCTS
ICSLYQLENYCGGGGGSGGGGSGGGGSGGGGSERKSSVESPPSPA
PPVAGPSVFLFPPKPKDTLMISRTPEVTCVVVDVSQEDPEVQFNW
YVDGVEVHNAKTKPREEQFNSTFRVVSVLTVLHQDWLNGKEYKCK
VSNKGLPSSIEKTISKTKGQPREPQVYTLPPSREEMTKNQVSLTC
LVKGFYPSDIAVEWESNGQPENNYKTTPPMLDSDGSFFLYSKLTV
DKSRWQQGNVFSCSVMHEALHNHYTQKSLSLSLG

In one embodiment, the fusion protein of the present invention exists in the form of a dimer. In certain embodiments, the dimer is a homodimer, wherein the amino acid sequences of the two fusion proteins constituting the dimer are identical. In certain embodiments, the dimer is a heterodimer, wherein the amino acid sequences of the two fusion proteins constituting the dimer are different.

Therefore, the present invention provides a dimer comprising the aforementioned fusion protein.

In another aspect, the present invention provides a pharmaceutical composition comprising: the fusion protein of the present invention or a dimer thereof; and optionally at least one pharmaceutically acceptable excipient.

In another aspect, the present invention provides a nucleic acid molecule comprising a nucleotide sequence encoding the fusion protein of the present invention.

In yet another aspect, the present invention provides a recombinant vector comprising the above nucleic acid molecule.

In yet another aspect, the present invention provides a recombinant cell comprising the above nucleic acid molecule or recombinant vector.

In still another aspect, the present invention provides use of the fusion protein or dimer or pharmaceutical composition of the present invention in the preparation of a medicament for treating diabetes, obesity or metabolic syndrome.

In yet another aspect, the present invention provides a method of treating a patient with diabetes, obesity or metabolic syndrome, comprising administering to the patient in need thereof a therapeutically effective amount of the fusion protein or dimer or pharmaceutical composition of the present invention.

### BRIEF DESCRIPTION OF DRAWINGS

FIG. 1 shows pharmacodynamic data of an exemplary fusion protein of the present invention in a streptozotocin (STZ) treated mouse diabetes model.

### DETAILED DESCRIPTION

### Terminology

As used herein, the term “insulin analogue” refers to a protein that has insulin activity and is able to bind to and activate the insulin receptor, leading to a reduction in blood glucose levels and/or inhibition of hepatic glucose output, and its characteristics can be tested and measured using known techniques (such as those shown in the studies described below).

As used herein, the terms “insulin A chain” and “insulin B chain” refer to the A and B chains of the human insulin molecule (CAS No. 11061-68-0) whose native wild-type sequence is known. The human insulin A chain consists of 21 amino acids, called A₁-A₂₁ in the art, and has the sequence of:

(SEQ ID NO: 21)
GIVEQCCTSICSLYQLENYCN

The human insulin B chain consists of 30 amino acids, called B₁-B₃₀ in the art, and has the sequence of:

(SEQ ID NO: 22)
FVNQHLCGSHLVEALYLVCGERGFFYTPKT

As used herein, “insulin analog”, “insulin receptor agonist” are used interchangeably herein.

As used herein, “peptide linker”, “linker sequence”, and “linker” are used interchangeably herein. In certain embodiments, it refers to one or more amino acids that connect the insulin B chain analog and the insulin A chain analog to provide sufficient flexibility to achieve a structure necessary for binding to the insulin receptor. In certain embodiments, it refers to one or more amino acids that connect the insulin analog and the immunoglobulin Fc domain to provide sufficient mobility. Examples of suitable linkers include single glycine (Gly), serine (Ser) or valine (Val) residue, and the identity and sequence of amino acid residues in the linker may vary depending on the type of secondary structural elements that need to be achieved in the linker.

As used herein, EC₅₀ refers to a concentration of insulin-Fc fusion protein where a half-maximal response for insulin receptor binding in vitro is observed (e.g., the concentration at which insulin receptor binding is halfway achieved).

As used herein, IC₅₀ refers to a concentration of insulin-Fc fusion protein, wherein a given biological function or biochemical process (e.g., binding) is inhibited by half. In some embodiments, IC₅₀ refers to a concentration of insulin-Fc fusion protein, wherein the binding of insulin to the human insulin receptor is reduced by half.

As used herein, the term “fusion protein”, e.g., “insulin-Fc fusion” protein refers to a protein comprising one or more domains, e.g., typically from different sources (e.g., different proteins, polypeptides, cells, etc.), the domains being connected by a polypeptide linker. In some embodiments, the fusion protein is recombinantly produced. In some embodiments, the domains of the fusion protein are connected by connecting the gene sequences encoding each domain into a single nucleic acid molecule. In some embodiments, the insulin-Fc fusion protein is a protein, e.g., a single polypeptide, comprising an insulin polypeptide and an Fc fragment polypeptide, wherein the insulin and Fc fragment polypeptides are connected by a peptide linker to form a single polypeptide.

### Insulin Analogs

In the fusion protein of the present invention, the analogue of insulin B chain in the insulin analogue includes one or more modifications to human insulin B chain. Specifically, in order to reduce the tendency of partial dimerization of the insulin analog, the insulin B chain analog comprises one or more modifications at positions B₅, B₁₆, B₂₅ or B₂₇₋₃₀ of the B chain of the human insulin molecule, which are respectively represented as positions X₁, X₂, X₃ and X₄₋₇ in SEQ ID NO: 1. For example: X₁ (which corresponds to B₅ in the B chain of the human insulin molecule) may be modified to A; X₅ (which corresponds to B₁₆ in the B chain of the human insulin molecule) may be modified to A; X₃ (which corresponds to B₂₅ in the B chain of the human insulin molecule) may be modified to T, I or F; X₄ (which corresponds to B₂₇ in the B chain of the human insulin molecule) may be deleted or modified to E or D; X₅ (which corresponds to B₂₈ in the B chain of the human insulin molecule) may be deleted or modified to E; and X₆ (which corresponds to B₂₉ in the B chain of the human insulin molecule) may be deleted or modified to E, and X₇ (which corresponds to B₃₀ in the B chain of the human insulin molecule) may be deleted.

In certain embodiments, the analog of the insulin B chain in the insulin receptor partial agonist portion comprises one or more modifications to the human insulin B chain, including modifications at positions X₁, X₂, X₃ and X₄₇ of SEQ ID NO: 1 to modulate the efficacy (e.g. reduce the affinity with the insulin receptor) and improve protein expression. In a preferred embodiment, X₂ is E.

In certain embodiments, the analog of the insulin A chain in the insulin receptor partial agonist includes one or more modifications to the amino acid sequence of the human insulin A chain to improve chemical and physical stability, modulate efficacy, eliminate deamidation, and/or enhance expression. In the modifications at positions X₈, X₉, X₁₀ and X₁₁ in SEQ ID NO: 2, X₈ (which corresponds to A₃ in the A chain of the human insulin molecule) is modified to A; X₉ (which corresponds to A₅ in the A chain of the human insulin molecule) is modified to A; X₁₀ (which corresponds to A₅ in the A chain of the human insulin molecule) is modified to A, K or F; and X₁₁ (which corresponds to A₂₁ in the A chain of the human insulin molecule) is modified to G, S or A. In a preferred embodiment, X₈ is A and X₄ is G.

In the fusion protein of the present invention, the C-terminal residue of the insulin B chain analog is directly fused to the N-terminal residue of the first peptide linker, and the C-terminal residue of the first peptide linker is directly fused to the N-terminal residue of the insulin B chain analog. The first peptide linker must provide sufficient flexibility for analogs of the insulin B chain and the insulin A chain to achieve the necessary structure for binding to the insulin receptor. In certain embodiments, the amino acid sequence of the first peptide linker is selected from the group consisting of: GGVGGG (SEQ ID NO: 16), GGGSGG (SEQ ID NO: 17), and GGGGGV (SEQ ID NO: 18)). Most preferably, the sequence of the first peptide linker is GGVGGG (SEQ ID NO: 16).

As described above, the C-terminal residue of the insulin receptor agonist portion of the fusion protein of the invention is fused to the N-terminal residue of the second peptide linker, and the C-terminal residue of the second peptide linker is fused directly to the N-terminal residue of the Fc portion. Preferably, the second peptide linker is enriched in glycine to provide sufficient conformational flexibility. Preferably, the second peptide linker is less than 30 amino acids in length. In certain preferred embodiments, the second peptide linker is 15 to 25 amino acids in length, wherein at least 50% of the amino acids are glycine residues. Preferred second peptide linker comprises the sequence of (GGGGX)n, where X is S or T, and n=2,3,4,5, and the most preferred second peptide linker has the amino acid sequence of SEQ ID NO: 4.

### Human IgG Fc Region

As used herein, the term “human IgG Fc region” has the meaning generally given to the term in the field of immunology. In particular, the term refers to a human IgG antibody fragment obtained by removing two antigen binding regions (Fab fragments) from an antibody. Specifically, the Fc region includes the CH2 and CH3 constant region domains of the antibody, and may also include some or all of the hinge regions.

As described above, in certain embodiments of the fusion protein of the present invention, the human IgG Fc region comprises a fragment of the constant region from one heavy chain of an IgG antibody, and in other embodiments, the human IgG Fc region comprises fragments of the constant regions from two heavy chains of an IgG antibody. In this embodiment, the constant regions of the two heavy chains are associated with each other by non-covalent interactions.

Human IgG isotypes (subclasses of mature gamma globulin G class antibodies; IgG1, IgG2, IgG3, and IgG4) have different structures and exhibit different biological functions known as “effector function”, such as antibody-dependent cellular cytotoxicity (ADCC, e.g. IgG1 and IgG3), antibody-dependent cellular phagocytosis (ADCP, e.g. IgG1, IgG2, IgG3, and IgG4), and complement-dependent cytotoxicity (CDC, e.g. IgG1, IgG3). Among the various subclasses, IgG1 and IgG3 have high relative affinities for Fcγ receptors (e.g. FcγRI, FcγRIIa/b/c, FcγRIIIa/b), low affinity for IgG2 (limited to FcγRIIa 131H polymorphism), and IgG4 has only measurable affinity for FcγRI. The fusion proteins of the present invention may contain Fc regions from any IgG subclass, with IgG2 and IgG4 being preferred because they have lower receptor binding and effector function activities than IgG1 and IgG3.

As used herein, the term human IgG Fc region also includes versions of such antibody fragments that have been modified, extended and/or truncated to alter properties or characteristics, such as complement and/or Fc receptor binding function, effector function, disulfide bond formation, glycosylation, antibody-dependent cell-mediated cytotoxicity (ADCC), manufacturability and/or stability. For example, the human IgG Fc region of the fusion protein of the present can be modified to reduce or remove N-linked glycosylation sites, which will reduce Clq binding affinity and cytotoxicity, affect conformational stability and in vivo clearance, and/or alter effector function.

In the human IgG Fc region of the fusion protein of the present invention the disulfide bonds of the hinge region may also be removed to simplify disulfide-mediated Fc dimerization. Other examples include phosphorylation, sulfation, acylation, glycosylation, methylation, acetylation, amidation and/or modifications capable of producing heterodimeric molecules. Techniques for altering the structure and properties of the human IgG Fc region of the IgG subclass are known in the art.

Regardless of the final structure of the fusion protein, the human IgG Fc region must be used to extend the in vivo plasma half-life of the insulin analog, while minimizing any Fc effector function is important. In addition, the fused insulin receptor agonist must maintain the ability to bind to and activate the insulin receptor to result in a reduction in blood glucose levels and/or inhibition of hepatic glucose output.

A preferred human IgG Fc region is an IgG2 Fc region that is modified to further reduce the effector function, as described in US20070148167, wherein modifications of H 268Q, V309L, A330S, and P 330S (EU numbering) are comprised and its hinge region 4 has disulfide bond deletion, and has modifications of C219S, C220S, C223S, and C226S, as shown in SEQ ID NO: 5, wherein X₁₆ is S; X₁₇ is C; X₁₈ is S; X₁₉ is S; X₂₀ is Q; X₂₁ is L; X₂₂ is S; X₂₃ is S; X₂₄ is L; X₂₅ is K.

Another preferred human IgG Fc region is an IgG4 Fc region that is modified to further reduce effector function and promote homodimer formation, as shown in SEQ ID NO: 6.

The disclosure of the present invention provides a polynucleotide encoding any one of the fusion proteins, a vector comprising the recombinant nucleic acid sequence disclosed herein and an engineered eukaryotic cell comprising the vector disclosed herein, for example, transfected with the recombinant nucleic acid sequence (e.g., mRNA, cDNA, DNA) encoding the insulin-Fc fusion protein described herein.

### Expression and Purification of Fusion Proteins

The fusion protein of the present invention is produced in a mammalian cell expression system using a CHO glutamine synthetase (GS) knockout (GSKO) cell line. GS gene knockout achieves tighter selection stringency by eliminating endogenous GS background activity that allows low-productivity or non-productive cells to survive under selection conditions. The gene encoding the fusion protein is subcloned into an expression plasmid containing glutamine synthetase (GS). The cDNA sequence encoding the fusion protein is fused in frame with the coding sequence of a signal peptide that enhances the secretion of the fusion protein into the cell culture medium. Expression is driven by a cytomegalovirus (CMV) promoter. Electroporation and an appropriate amount of the recombinant expression plasmid are used to stably transfect CHO GSKO cells.

Transfected cells undergo bulk selection in glutamine-free medium. The transfected pool is plated at low density to allow close-to-clonal outgrowth of stably expressing cells. Master wells are screened for fusion protein expression and scaled up in serum-free suspension culture to be used for production.

The fusion protein secreted into the culture medium can be purified by protein A affinity chromatography followed by size exclusion chromatography following standard chromatography techniques. In brief, fusion protein selecting protein A (GE) that has been balanced with a pH 7.4 phosphate buffered saline is used to capture the fusion protein from the clarified culture medium. After a washing step with a pH 7.4 phosphate buffered saline, the bound fusion protein is eluted with a pH 3.0 glycine. The fractions containing the fusion protein are combined and neutralized by adding 1/10 volume of a 1M pH 9.0 Tris.Soluble aggregates and multimers can be effectively removed by conventional techniques, including size exclusion, hydrophobic interaction, or ion exchange chromatography. Fractions containing monomeric fusion proteins (non-covalently linked homodimers) as determined by size exclusion chromatography are combined, sterilized by filtration, and stored.

### EXAMPLES

The following examples will further illustrate the present invention in conjunction with the accompanying drawings.

The experimental methods used in the following examples are conventional methods unless otherwise specified.

The materials, reagents, etc. used in the following examples, unless otherwise specified, can all be obtained from commercial sources.

### Example 1: (B27E+A3-A+A21G)

A fusion protein of the present invention has an amino acid sequence of:

(SEQ ID NO: 8)
FVNQHLCGSHLVEALYLVCGERGFFYEPKTGGVGGGGIAEQCCTS
ICSLYQLENYCGGGGGSGGGGSGGGGSGGGGSERKSSVESPPSPA
PPVAGPSVFLFPPKPKDTLMISRTPEVTCVVVDVSQEDPEVQFNW
YVDGVEVHNAKTKPREEQFNSTFRVVSVLTVLHQDWLNGKEYKCK
VSNKGLPSSIEKTISKTKGQPREPQVYTLPPSREEMTKNQVSLTC
LVKGFYPSDIAVEWESNGQPENNYKTTPPMLDSDGSFFLYSKLTV
DKSRWQQGNVFSCSVMHEALHNHYTQKSLSLSLG

### Example 2: (A3−A+A21G)

A fusion protein of the present invention has an amino acid sequence of:

(SEQ ID NO: 9)
FVNQHLCGSHLVEALYLVCGERGFFYTPKTGGVGGGGIAEQCCTS
ICSLYQLENYCGGGGGSGGGGSGGGGSGGGGSERKSSVESPPSPA
PPVAGPSVFLFPPKPKDTLMISRTPEVTCVVVDVSQEDPEVQFNW
YVDGVEVHNAKTKPREEQFNSTFRVVSVLTVLHQDWLNGKEYKCK
VSNKGLPSSIEKTISKTKGQPREPQVYTLPPSREEMTKNQVSLTC
LVKGFYPSDIAVEWESNGQPENNYKTTPPMLDSDGSFFLYSKLTV
DKSRWQQGNVFSCSVMHEALHNHYTQKSLSLSLG

### Example 3: (A5−A+A21G)

A fusion protein of the present invention has an amino acid sequence of:

(SEQ ID NO: 10)
FVNQHLCGSHLVEALYLVCGERGFFYTPKTGGVGGGGIVEACCTS
ICSLYQLENYCGGGGGSGGGGSGGGGSGGGGSERKSSVESPPSPA
PPVAGPSVFLFPPKPKDTLMISRTPEVTCVVVDVSQEDPEVQFNW
YVDGVEVHNAKTKPREEQFNSTFRVVSVLTVLHQDWLNGKEYKCK
VSNKGLPSSIEKTISKTKGQPREPQVYTLPPSREEMTKNQVSLTC
LVKGFYPSDIAVEWESNGQPENNYKTTPPMLDSDGSFFLYSKLTV
DKSRWQQGNVFSCSVMHEALHNHYTQKSLSLSLG

### Example 4: (A8−A+A21G)

A fusion protein of the present invention has an amino acid sequence of:

(SEQ ID NO: 11)
FVNQHLCGSHLVEALYLVCGERGFFYTPKTGGVGGGGIVEQCCAS
ICSLYQLENYCGGGGGSGGGGSGGGGSGGGGSERKSSVESPPSPA
PPVAGPSVFLFPPKPKDTLMISRTPEVTCVVVDVSQEDPEVQFNW
YVDGVEVHNAKTKPREEQFNSTFRVVSVLTVLHQDWLNGKEYKCK
VSNKGLPSSIEKTISKTKGQPREPQVYTLPPSREEMTKNQVSLTC
LVKGFYPSDIAVEWESNGQPENNYKTTPPMLDSDGSFFLYSKLTV
DKSRWQQGNVFSCSVMHEALHNHYTQKSLSLSLG

### Example 5: (B5−A+A21G)

A fusion protein of the present invention has an amino acid sequence of:

(SEQ ID NO: 12)
FVNQALCGSHLVEALYLVCGERGFFYTPKTGGVGGGGIVEQCCTS
ICSLYQLENYCGGGGGSGGGGSGGGGSGGGGSERKSSVESPPSPA
PPVAGPSVFLFPPKPKDTLMISRTPEVTCVVVDVSQEDPEVQFNW
YVDGVEVHNAKTKPREEQFNSTFRVVSVLTVLHQDWLNGKEYKCK
VSNKGLPSSIEKTISKTKGQPREPQVYTLPPSREEMTKNQVSLTC
LVKGFYPSDIAVEWESNGQPENNYKTTPPMLDSDGSFFLYSKLTV
DKSRWQQGNVFSCSVMHEALHNHYTQKSLSLSLG

### Example 6: (B16−A+A3−A+A21G)

A fusion protein of the present invention has an amino acid sequence of:

(SEQ ID NO: 13)
FVNQHLCGSHLVEALALVCGERGFFYTPKTGGVGGGGIAEQCCTS
ICSLYQLENYCGGGGGSGGGGSGGGGSGGGGSERKSSVESPPSPA
PPVAGPSVFLFPPKPKDTLMISRTPEVTCVVVDVSQEDPEVQFNW
YVDGVEVHNAKTKPREEQFNSTFRVVSVLTVLHQDWLNGKEYKCK
VSNKGLPSSIEKTISKTKGQPREPQVYTLPPSREEMTKNQVSLTC
LVKGFYPSDIAVEWESNGQPENNYKTTPPMLDSDGSFFLYSKLTV
DKSRWQQGNVFSCSVMHEALHNHYTQKSLSLSLG

### Example 7: (B25−T+B27−E A3−A+A21G)

A fusion protein of the present invention has an amino acid sequence of:

(SEQ ID NO: 14)
FVNQHLCGSHLVEALYLVCGERGFTYEPKTGGVGGGGIAEQCCTS
ICSLYQLENYCGGGGGSGGGGSGGGGSGGGGSERKSSVESPPSPA
PPVAGPSVFLFPPKPKDTLMISRTPEVTCVVVDVSQEDPEVQFNW
YVDGVEVHNAKTKPREEQFNSTFRVVSVLTVLHQDWLNGKEYKCK
VSNKGLPSSIEKTISKTKGQPREPQVYTLPPSREEMTKNQVSLTC
LVKGFYPSDIAVEWESNGQPENNYKTTPPMLDSDGSFFLYSKLTV
DKSRWQQGNVFSCSVMHEALHNHYTQKSLSLSLG

### Example 8: (des B27−30+A3−A+A21G)

A fusion protein of the present invention has an amino acid sequence of:

(SEQ ID NO: 15)
FVNQHLCGSHLVEALYLVCGERGFFYGGVGGGGIAEQCCTSICSL
YQLENYCGGGGGSGGGGSGGGGSGGGGSERKSSVESPPSPAPPVA
GPSVFLFPPKPKDTLMISRTPEVTCVVVDVSQEDPEVQFNWYVDG
VEVHNAKTKPREEQFNSTFRVVSVLTVLHQDWLNGKEYKCKVSNK
GLPSSIEKTISKTKGQPREPQVYTLPPSREEMTKNQVSLTCLVKG
FYPSDIAVEWESNGQPENNYKTTPPMLDSDGSFFLYSKLTVDKSR
WQQGNVFSCSVMHEALHNHYTQKSLSLSLG

Meanwhile, the unmodified Insulin-Fc fusion protein used in the present invention has an amino acid sequence of:

(SEQ ID NO: 23)
FVNQHLCGSHLVEALYLVCGERGFFYEPKTGGVGGGGIVEQCCTS
ICSLYQLENYCNGGGGSGGGGSGGGGSGGGGSERKSSVESPPSPA
PPVAGPSVFLFPPKPKDTLMISRTPEVTCVVVDVSQEDPEVQFNW
YVDGVEVHNAKTKPREEQFNSTFRVVSVLTVLHQDWLNGKEYKCK
VSNKGLPSSIEKTISKTKGQPREPQVYTLPPSREEMTKNQVSLTC
LVKGFYPSDIAVEWESNGQPENNYKTTPPMLDSDGSFFLYSKLTV
DKSRWQQGNVFSCSVMHEALHNHYTQKSLSLSLG

### Experimental Examples

### Experimental Example 1: Detection of Purity and Thermal Stability of Fusion Proteins

The purity of the fusion protein monomers were detected by Size Exclusion Chromatography-High Performance Liquid Chromatography (SEC-HPLC). Specifically, after the protein solution was diluted to 1 mg/ml, it was detected using Size Exclusion Chromatography—High Performance Liquid Chromatography column MAbPac™ SEC1 (7.8 mm×300 mm, Thermo Fisher Scientific, Waltham, MA, USA) with a sample volume of 10 μL. The mobile phase was a 20 mM phosphate buffer solution (pH 7.0, Sinopharm Co., Ltd., Shanghai, China) containing 150 mM sodium chloride, and the flow rate was 0.6 mL/min. Analytical SEC was performed on an Agilent 1100 (Agilent Technologies, Inc., Santa Clara, CA, USA) system for 20 minutes, and the signal value was obtained using a 280 nm UV detector.

This accelerated test was to examine the purity and aggregate changes of the fusion protein at high temperature. The storage condition was heating at 60° C. for 1 hour, and SEC-HPLC was used to determine the purity change after heating. Table 1 summarizes the changes in monomer purity of the fusion protein at the time of zero and after heating at 60° C. for 1 h, wherein unmodified Insulin-Fc represents an Fc fusion protein (SEQ: NO.23) with the same structure as the example protein and unmodified insulin amino acids. The changes in monomer purities of the fusion proteins of the examples of the present invention after heating at 60° C. for 1 hour compared with that at the time of zero were between 2.9% and 8.8%. This result supports that the fusion proteins of the examples of the present invention have high thermal stability at 60° C.

TABLE 1
Monomer purity and thermal stability detection
of insulin-Fc fusion protein

| | | | | Monomer |
|---|---|---|---|---|
| | | | | Purity |
| | | Time of | 60 ° C. | Change |
| | Fusion Protein | Zero | 1 h | Amount |
| | Unmodified Insulin-Fc | 93.6% | 83.4% | 10.2% |
| | (SEQ ID NO: 23) | | | |
| Example | B₂₇E + A₃-A + A₂₁G | 98.2% | 91.5% | 6.7% |
| 1 | | | | |
| Example | A₃ − A + A₂₁G | 93.6% | 87.9% | 5.7% |
| 2 | | | | |
| Example | A₅ − A + A₂₁G | 97.9% | 93.0% | 4.9% |
| 3 | | | | |
| Example | A₈ − A + A₂₁G | 96.6% | 92.2% | 4.4% |
| 4 | | | | |
| Example | B₅ − A + A₂₁G | 100% | 97.1% | 2.9% |
| 5 | | | | |
| Example | B₁₆ − A + A₃ − A + A₂₁G | 95.6% | — | — |
| 6 | | | | |
| Example | B₂₅ − T + B₂₇ − E + A₃ − | 94.8% | 90.8% | 4.0% |
| 7 | A + A₂₁G | | | |
| Example | desB_{27 - 30} + A₃ − A + A21G | 95.8% | 87% | 8.8% |
| 8 | | | | |

“—” means not detected

### Experimental Example 2: Detection of the Binding of Insulin Fusion Protein to Receptor by Enzyme-Linked Immunosorbent Assay (ELISA)

Detection Procedure: the human insulin receptor extracellular domain protein (hIRB-ECD, Sino Biological Inc., Beijing, China) was diluted to 5 μg/mL with PBS at pH 7.2, and a 96-well high-adsorption U-shaped plate (Greiner Bio-One North America Inc., Monroe, NC, USA) was coated at 50 μL/well, incubated overnight at 4° C., and washed with PBS 3 times. The 96-well plate was blocked with a blocking solution (1% casein, Thermo Fisher Scientific, Waltham, MA, USA) at 200 μL/well, left at room temperature for 1.5 hours, and washed with 0.1% PBST 3 times. Samples including insulin-Fc fusion protein or Fc isotype control antibody (Thermo Fisher Scientific (Cat: 31154)) diluted gradiently with the blocking solution at a ratio of 1:3 (exemplary concentrations are: 10 μM, 3.3 μM, 1.1 μM, 0.37 UM, 0.12 μM, 0.041 μM, 0.014 μM, 0 μM) were added respectively at 50 μL/well, with 2 replicates set up for each well, and incubated at room temperature for 1 hour, and washed with 0.1% PBST 3 times. HRP Goat Anti Human IgG (H+L) (ABclonal Technology Co., Ltd., Wuhan, China) diluted with the blocking solution at 1:2000 was added at 50 μL/well, incubated at room temperature for 1 hour and washed with 0.1% PBST 3 times. TMB solution (Thermo Fisher Scientific) was added at 50 μL/well and incubated at room temperature for 5 minutes, and then 2M sulfuric acid was added at 50 μL/well to terminate the reaction. The absorbance value was read at a wavelength of 450 nm on a microplate reader SpectraMax M5e (Molecular Devices, LLC, San Jose, Calif., USA), and data analysis was performed using GraphPad Prism 9 (GraphPad Software Inc., San Diego, Calif., USA).

Table 2 summarizes the EC₅₀ values of insulin-Fc fusion protein binding to insulin receptor. The results show that the fusion proteins of the examples of the present invention have reduced hIRB binding ability compared with unmodified Insulin-Fc.

TABLE 2
EC₅₀ values of insulin-Fc fusion protein and insulin receptor

| | Insulin Fc Fusion Protein | EC₅₀(μM) |
|---|---|---|
| | Unmodified Insulin-F c | 0.01180 |
| | (SEQ ID NO: 23)) | |
| Example 1 | B₂₇ − E + A₃ − A + A₂₁G | 0.1599 |
| Example 2 | A₃ − A + A₂₁G | 0.2864 |
| Example 3 | A₅ − A + A₂₁G | 0.09159 |
| Example 4 | A₈ − A + A₂₁G | 0.06612 |
| Example 5 | B₅ − A + A₂₁G | 0.1596 |
| Example 6 | B₁₆ − A + A₃ − A + A₂₁G | 0.4452 |
| Example 7 | B₂₅ − T + B₂₇ − E + A₃ − A + | 0.6991 |
| | A₂₁G | |
| Example 8 | desB₂₇ − 30 + A₃ − A + A21G | 0.3140 |

### Experimental Example 3: Detection of the Affinity of Insulin-Fc Fusion Protein and Human Insulin-Like Growth Factor 1 Receptor (IGF-1R) by Biomembrane Interferometry

The affinity analysis of each fusion protein and IGF-1R-His tag (Sino Biological) was performed using a FortéBio Octet® (Pall FortéBio LLC, Fremont, CA, USA) instrument. The His-tagged IGF-1R protein (10 μg/mL) was captured by Ni-NTA probe (Pall FortéBio LLC), and the interaction activity with glargine insulin (Lantus, Sanofi), unmodified Insulin-Fc, and the fusion proteins of Example 1 and Example 2 at different concentrations of 500 nM, 250 nM, 125 nM, 62.5 nM, 31.25 nM, 15.6 nM, 7.8 nM, and 0 nM was detected in a 0.1% PBST universal buffer. The affinity constants of IGF-1R with various insulin fusion proteins were calculated based on the affinity and kinetics 1:1 binding mode fitting in Octet Analysis software.

Table 3 summarizes the binding and dissociation kinetic data, average Kₒₙ, K_{off} and apparent KD values of insulin-Fc fusion protein with IGF-1R. Studies have shown that IGF-1R is highly expressed in a variety of cancer tissues, and insulin can exert a pro-proliferative effect by binding to IGF-1R, and ideal insulin analogs should have similar or reduced IGF-1R binding affinity to unmodified insulin. The results in Table 3 show that the fusion proteins of Examples 1 and 2 of the present invention have reduced affinity for IGF-1R compared to glargine insulin (Lantus, Sanofi) and unmodified Insulin-Fc, indicating that the fusion proteins of the examples of the present invention do not have potential pro-proliferative risk.

TABLE 3
KD values of insulin-Fc fusion proteins and insulin-like
growth factor 1 receptor (IGF-1R)

| | | Kₒₙ | K_{off} | KD |
|---|---|---|---|---|
| | Fusion Protein | (1/Ms) | (1/s) | (M) |
| | Lantus | 2.511 E+04 | 1.919 E−02 | 7.644 E−07 |
| | Insulin-Fc | 2.003 E+03 | 9.869 E−04 | 4.927 E−07 |
| | (SEQ ID NO: 23) | | | |
| Example | B27E + A₃ − A + | 5.182 E+02 | 3.364 E−03 | 6.492 E−06 |
| 1 | A₂₁G | | | |
| Example | A3 − A + A₂₁G | 2.225 E+02 | 6.673 E−04 | 2.999 E−06 |
| 2 | | | | |

### Experimental Example 4: Detection of the Binding Affinity of Insulin-Fc Fusion Proteins to IRA/IRB and IGF-1R by Scintillation Proximity Assay (SPA)

IRA, IRB, and IGF-1R binding assays were performed in SPA assays in a 96-well plate using cell membranes prepared from two CHO cells overexpressing hIRA and hIRB, respectively, and H19-7 cells overexpressing human IGF-1R. In a 96-well conical plate (Agilent), 25 μl of cell membranes of hIRA, hIRB, and IGF-1R overexpressing cell lines diluted with 1 M Trizma hydrochloride buffer (Sigma-Aldrich) were added to each well. The fusion proteins of examples and the natural recombinant human insulin (RHI, CAS No.: 11061-68-0, Yisheng Bio) were gradiently diluted in 1:4 with 1M Trizma buffer and added to the 96-well plate containing cell membranes at 25 μl/well, with 2 replicates set up for each well. In the IRA/IRB binding assay, 50 μl of ^{[125I]}-Insulin (PerkinElmer, Inc., Waltham, MA) with a final concentration of 100 μM was added to the above system. Similarly, in the IGF-1R binding assay, 50 μl of ^{[125I]}-IGF-1 (PerkinElmer, Inc.) was added at a final concentration of 100 μM. Subsequently, the 96-well plate was sealed with a sealing film and incubated on a shaker at room temperature for 1 hour. The above system was collected using a cell harvester into a 96-well-GF/C filter plate (PerkinElmer, Inc.) that had been soaked in 0.5% BSA solution in advance, washed 6 times with 1 M Trizma buffer, and dried in a 50° C. oven for 1 hour. After that, 50 μL of scintillation fluid (PerkinElmer, Inc.) was added to each well and read using a MicroBeta² (PerkinElmer, Inc.) instrument. The IC₅₀ value was calculated using a four-parameter fit using a nonlinear model in GraphPad Prism 5.0 (San Diego, CA) and quantified by the percentage (%) of the maximum receptor binding activity relative to natural recombinant human insulin (RHI).

Table 4 summarizes the IC₅₀ values of insulin-Fc fusion proteins and natural recombinant human insulin with hIRA, hIRB and hIGF-1R and the maximum receptor binding activity percentage relative to natural insulin. The results show that the insulin-Fc fusion proteins of Examples 1 and 2 of the present invention have significantly reduced binding affinity to IRA, IRB and IGF-1R compared to natural recombinant human insulin and unmodified Insulin-Fc fusion protein, and the maximum receptor binding activity of Examples 1 and 2 with IRA, IRB and IGF-1R is significantly lower than that of recombinant human insulin.

TABLE 4
IC₅₀ values of insulin-Fc fusion proteins with IRA, IRB and IGF-1R and the maximum
receptor binding activity percentage relative to recombinant human insulin

| | hIRA | | hIRB | | hIGF-1R | |
|---|---|---|---|---|---|---|
| | | Maximum | | Maximum | | Maximum |
| | | Receptor | | Receptor | | Receptor |
| | | Binding | | Binding | | Binding |
| | | Activity | | Activity | | Activity |
| | | (%) | | (%) | | (%) |
| | IC₅₀ | Relative | IC₅₀ | Relative | IC₅₀ | Relative |
| | (nM) | to RHI | (nM) | to RHI | (nM) | toRHI |
| RHI | 2.864 | 109.1 | 1.057 | 105.3 | 106.8 | 79.4 |
| Unmodified | 16.6 | 66.49 | 76.58 | 88.55 | 646.8 | 87.5 |
| Insulin-Fc (SEQ | | | | | | |
| ID NO: 23)) | | | | | | |
| Example 1 | 101.8 | 78.69 | 508.7 | 74.27 | >62500 | NA |
| Example 2 | 1402 | 54.97 | 1941 | 54.46 | >19000 | NA |

### Experimental Example 5: Study of Insulin-Fc Fusion Protein in STZ-Treated Diabetic Mouse Model

Normal C57BL/6 mice (SCXK (Jing) 2019-0008, weight 18-22 g) were adapted to the environment for about 1 week, fasted for 12 h overnight, and intraperitoneally injected with 60 mg/kg streptozotocin (STZ, Sigma-Aldrich), and then resumed feeding, and continued to be injected at the same time every day for 6 days. After 7 consecutive days of feeding, fasting blood glucose was measured using a blood glucose meter (Roche, Basel, Switzerland), and blood glucose was measured twice in a row. The model was successfully established when the blood glucose value was greater than 12 mmol/L each time. The mice were grouped according to the last weight and blood glucose for subsequent pharmacodynamic evaluation.

The random blood glucose of the mice with successful modeling was measured 1 h before administration for fine-tuning of grouping, and 300 nmol/kg of the insulin-Fc fusion protein of Example 1 and/or Example 2 prepared in sterile PBS was subcutaneously injected at a point behind the neck, and the blood glucose was measured at 2, 4, 6, 8 and 24 h after injection, respectively, and then the blood glucose was measured every 24 h until the modeling level was restored.

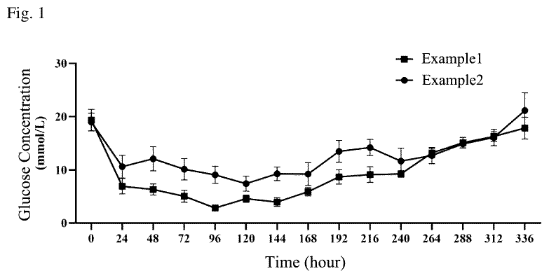

FIG. 1 shows the collected blood glucose data (mean±SEM, n=6). For a more intuitive display, the data within 0-24 h are not included in the FIGURE. The results show that the fusion protein of Example 1 can maintain blood glucose fluctuations at normal levels within 240 hours after administration and the mice have almost no hypoglycemia. The hypoglycemic effect can last for about 14 days, which is better than the fusion protein of Example 2 in terms of hypoglycemic effect and lasting time. The injection of unmodified Insulin-Fc fusion protein at 300 nmol/kg caused obvious hypoglycemia in mice within 48 h of administration, which is not shown in the FIGURE.

### Experimental Example 6: Pharmacokinetics Detection of Insulin-Fc Fusion Protein

Four four-week-old C57BL/6 mice (weight 18-22 g) were subcutaneously injected (s.c.) with 300 nmol/kg of Example 1, and blood was collected from the angular vein at 1 h, 2 h, 4 h, 8 h, 12 h, 24 h, 48 h, 96 h, 144 h, 196 h, and 240 h after injection (about 100 μL of blood was collected from each mouse). 10 μL of 100% EDTA was added to the blood and mixed, centrifuged at 4000 rpm*20 min, and the supernatant was collected.

ELISA was used to detect the concentration of insulin fusion protein in plasma, using the extracellular domain of human insulin receptor (hIRB, aa28-956) as the capture. A 96-well high-adsorption U-shaped plate was coated with 10 μg/ml hIRB (aa28-956) at 50 μL/well, incubated at 4° C. overnight and washed 3 times with PBS. The 96-well plate was blocked with 1% casein blocking solution at 200 μL/well, left at room temperature for 1.5 hours, and washed 3 times with 0.1% PBST. The fusion protein gradiently diluted at a ratio of 1:2 with the blocking solution (1% casein, Thermo Scientific Fisher) containing 5% mouse plasma was added, and plasma samples at different time points (n=4) were diluted 20 times with a 1% casein blocking solution at 50 μL/well, incubated at room temperature for 1.5 hours and washed with 0.1% PBST 3 times. The absorbance at 450 nm was detected using HRP Goat Anti Human IgG (H+L) (ABclonal Technology Co., Ltd.) diluted at 1:2000 in a 1% casein blocking buffer.

Table 5 shows the plasma concentration of the fusion protein of Example 1 at different time points, and Table 6 shows the pharmacokinetic parameters of the fusion protein of Example 1 obtained after non-compartmental analysis of the data in Table 5. The data show that the in vivo half-life of the fusion protein of Example 1 is relatively long, up to about 38 h, and compared with conventional insulin on the market, the bioavailability is increased and the duration of action in vivo is prolonged.

TABLE 5
Blood drug concentration of the fusion protein
of Example 1 in mice

| | Concentration (μg/mL) | | | |
|---|---|---|---|---|
| Time (h) | No. 1 | No. 2 | No. 3 | No. 4 |
| 1 | 3.960197 | 5.284146 | 5.17737 | 5.47976 |
| 2 | 11.20113 | 11.07989 | 8.223485 | 10.74274 |
| 4 | 15.66349 | 13.63894 | 16.51843 | 10.04345 |
| 8 | 18.42453 | 22.49654 | 22.33919 | 18.78905 |
| 12 | 53.37436 | 26.65293 | 26.94227 | 36.48718 |
| 24 | 43.2363 | 41.26902 | 39.39275 | 30.52699 |
| 48 | 37.82452 | 37.66351 | 23.37041 | 35.62251 |
| 96 | 9.2106 | 8.047675 | 8.091521 | 5.826623 |
| 144 | 4.530268 | 3.023386 | 4.233619 | 7.249348 |
| 196 | 2.550186 | 1.957366 | 4.381304 | 1.430619 |
| 240 | 0.06419 | 0.16382 | 1.553453 | 0.781461 |
| 288 | N/A | N/A | 0.14992 | 0.439542 |

TABLE 6
shows PK parameters of the fusion protein of Example 1 in mice.

| PK Parameters | Results |
|---|---|
| T_{1/2} (h) | 38.0 |
| Tₘₐₓ (h) | 24 |
| Cₘₐₓ (μg/mL) | 38.606 |
| AUCₗₐₛₜ (μg*h/mL) | 3105.464 |
| CL_F(nmol/(μg*h/mL)/kg) | 0.00965 |
| MRTₗₐₛₜ (h) | 58.484 |

Abbreviations:
T_{1/2}-half-life, Cmax-maximum concentration, Tmax-time at maximum concentration, AUCₗₐₛₜ-area under curve from 0 to administration point, CL_F-clearance rate, MRTₗₐₛₜ-average residence time from 0 to administration point.

1. A fusion protein comprising:
a) an insulin analog having a structure represented by the formula of Z₁-Z₂-Z₃, wherein:
Z₁ is an insulin B chain analog comprising the amino acid sequence of:
(SEQ ID NO: 1)
FVNQX₁LCGSHLVEALX₂LVCGERGFX₃YX₄X₅X₆X₇
wherein X₁ is H or A, X₂ is Y, A or E, X₃ is T, I, For H, X₄ is E, D, T or absent,
X₅ is P, E or absent, X₆ is K, E or absent, and X₇ is T or absent;
Z₂ is a first peptide linker;
Z₃ is an insulin A chain analog comprising the amino acid sequence of:
(SEQ ID NO: 2)
GIX₈EX₉CCX₁₀SICSLYQLENYCX₁₁
wherein X₈ is A, L or V; X₉ is Q or A; X₁₀ is T, A, For K; X₁₁ is G, S or N;
b) a second peptide linker; and
c) a human IgG Fc region or a mutant thereof;
wherein the C-terminal residue of the insulin analog is directly fused to the N-terminal residue of the second peptide linker, and the C-terminal residue of the second peptide linker is directly fused to the N-terminal residue of the human IgG Fc region or a mutant thereof.

2. The fusion protein according to claim 1, wherein in the insulin analogue,
in Z₁, X₁ is A, X₂ is Y, X₃ is F, X₄ is T, X₅ is P, X₆ is K, and X₇ is T; or
in Z₁, X₁ is H, X₂ is A, X₃ is F, X₄ is T, X₅ is P, X₆ is K, and X₇ is T; or
in Z₁, X₁ is H, X₂ is Y, X₃ is T or I, X₄ is E, X₅ is P, X₆ is K, and X₇ is T; or
in Z₁, X₁ is H; X₂ is Y; X₃ is F; X₄ is E or D; X₅ is P; X₆ is K; and X₇ is T; or
in Z₁, X₄-X₇ are absent.

3. The fusion protein according to claim 1, wherein in the insulin analogue,
in Z₃, X₈ is A or L, X₉ is A, X₁₀ is T, and X₁₁ is G; or
in Z₃, X₈ is V, X₉ is Q, X₁₀ is T, and X₁₁ is G; or
in Z₃, X₈ is V, X₉ is Q, X₁₀ is A, K or F, and X₁₁ is G; or
in Z₃, X₈ is A or L, X₉ is Q, X₁₀ is A or K, and X₁₁ is G; or

4. The fusion protein according to claim 1, wherein in the insulin analogue,
in Z₁, X₁ is H, X₂ is Y, X₃ is F, X₄ is E or D, X₅ is P, X₆ is K, and X₇ is T; and in Z₃, X₈ is A; X₁₁ is G; or
in Z₁, X₁ is H, X₂ is Y, X₃ is F, X₄ is T, X₅ is P, X₆ is K, and X₇ is T; and in Z₃, X₈ is A or L, and X₁₁ is G; or
in Z₁, X₁ is H, X₂ is Y, X₃ is F, X₄ is T, X₅ is P, X₆ is K, and X₇ is T; and in Z₃, X₉ is A, and X₁₁ is G; or
in Z₁, X₁ is H, X₂ is Y, X₃ is F, X₄ is T, X₅ is P, X₆ is K, and X₇ is T; and in Z₃, X₁₀ is A, K or F, and X₁₁ is G; or
in Z₁, X₁ is H, X₂ is A, X₃ is F, X₄ is T, X₅ is P, X₆ is K, X₇ is T; and in Z₃, X₈ is A, and X₁₁ is G; or
in Z₁, X₄-X₇ are absent; and in Z₃, X₈ is A, and X₁₁ is G.

5. The fusion protein according to claim 1, wherein Z₁-Z₂-Z₃ comprises the amino acid sequence of:
(SEQ ID NO: 19)
FVNQX₁LCGSHLVEALX₂LVCGERGFX₃YX₄X₅X₆X₇GGVGGGGI
X;EX₉CCX₁₀SICSLYQLENYCX₁₁
wherein X₁ is H or A; X₂ is Y, A or E; X₃ is T, I, For H; X₄ is E, D, T or absent; X₅ is P, E or absent; X₆ is K, E or absent; X₇ is T or absent; X₈ is A, L or V; X₉ is Q or A; X₁₀ is T, A, For K; X₁₁ is G, S or N,
preferably, Z₁-Z₂-Z₃ has the amino acid sequence of:
(SEQ ID NO: 3)
FVNQHLCGSHLVEALYLVCGERGFFYEPKTGGVGGGGIAEQCCTS
ICSLYQLENYCG.

6. The fusion protein according to claim 1, wherein the first peptide linker comprises an amino acid sequence selected from the group consisting of GGVGGG (SEQ ID NO: 16), GGGSGG (SEQ ID NO: 17), and GGGGGV (SEQ ID NO: 18), and/or
the second peptide linker comprises a peptide having the sequence of [GGGGX]n, wherein X is S or T; and wherein n is 2, 3, 4 or 5,
preferably, the second peptide linker comprises the amino acid sequence of:
(SEQ ID NO: 20)
GGGGX₁₂GGGGX₁₃GGGGX₁₄GGGGX₁₅
wherein X₁₂ is S or T; X₁₃ is S or T; X₁₄ is S or T; X₁₅ is S or T,
more preferably, the second peptide linker has the amino acid sequence of:
(SEQ ID NO: 4)
GGGGSGGGGSGGGGSGGGGS.

7. The fusion protein according to claim 1, wherein the human IgG Fc region is an Fc region derived from a IgG2 or IgG4 antibody, preferably an Fc region derived from a IgG2 antibody, more preferably comprising the amino acid sequence of:
(SEQ ID NO: 5)
ERKX₁₆X₁₇VEX₁₈PPX₁₉PAPPVAGPSVFLFPPKPKDTLMISRTPE
VTCVVVDVSX₂₀EDPEVQFNWYVDGVEVHNAKTKPREEQFNSTFR
VVSVLTVX₂₁HQDWLNGKEYKCKVSNKGLPX₂₂X₂₃IEKTISKTKG
QPREPQVYTLPPSREEMTKNQVSLTCLVKGFYPSDIAVEWESNG
QPENNYKTTPPMLDSDGSFFLYSKLTVDKSRWQQGNVFSCSVMH
EALHNHYTQKSLSLSX₂₄GX₂₅
wherein X₁₆ is S or C; X₁₇ is S or C; X₁₈ is S or C; X₁₉ is S or C; X₂₀ is Q or H; X₂₁ is L or V; X₂₂ is S or A; X₂₃ is S or P; X₂₄ is L or P; X₂₅ is K or absent,
still more preferably, the human IgG Fc region comprises the amino acid sequence of:
(SEQ ID NO: 6)
ESKYGPPSPPSPAPEAAGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLG.

8. The fusion protein according to claim 1, wherein the fusion protein comprises the amino acid sequence of:
(SEQ ID NO: 7)
FVNQX₁LCGSHLVEALX₂LVCGERGFX₃YX₄X₅X₆X₇GGVGGGGI
X₈EX₉CCX₁₀SICSLYQLENYCX₁₁GGGGX₁₂GGGGX₁₃GGGGX₁₄
GGGGX₁₅ERKX₁₆X₁₇VEX₁₈PPX₁₉PAPPVAGPSVFLFPPKPK
DTLMISRTPEVTCVVVDVSX₂₀EDPEVQFNWYVDGVEVHNAKTKP
REEQFNSTFRVVSVLTVX₂₁HQDWLNGKEYKCKVSNKGLPX₂₂X₂
3IEKTISKTKGQPREPQVYTLPPSREEMTKNQVSLTCLVKGFYPS
DIAVEWESNGQPENNYKTTPPMLDSDGSFFLYSKLTVDKSRWQQG
NVFSCSVMHEALHNHYTQKSLSLSX₂₄GX₂₅
wherein
X₁ is H or A;
X₂ is Y, A or E;
X₃ is T, I, F or H;
X₄ is E, D, T or absent;
X₅ is P, E or absent;
X₆ is K, E or absent;
X₇ is T or absent;
X₈ is A, L or V;
X₉ is Q or A;
X₁₀ is T, A, F or K;
X₁₁ is G, S or N;
X₁₂ is S or T;
X₁₃ is S or T;
X₁₄ is S or T;
X₁₅ is S, or T;
X₁₆ is S or C;
X₁₇ is S or C;
X₁₈ is S or C;
X₁₉ is S or C;
X₂₀ is Q or H;
X₂₁ is L or V;
X₂₂ is S or A;
X₂₃ is S or P;
X₂₄ is L or P;
X₂₅ is K or absent.

9. The fusion protein according to claim 1, wherein the fusion protein comprises an amino acid sequence selected from the group consisting of SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

10. A protein dimer comprising the fusion protein according to any one of claims 1 to 9.

11. A pharmaceutical composition comprising the fusion protein according to any one of claims 1 to 9 or the protein dimer according to claim 10, and optionally at least one pharmaceutically acceptable excipient.

12. A nucleic acid molecule comprising a nucleotide sequence encoding the fusion protein according to any one of claims 1 to 9 or the protein dimer according to claim 10.

13. A recombinant vector comprising the nucleic acid molecule according to claim 12.

14. A recombinant cell comprising the nucleic acid molecule according to claim 12 or the recombinant vector according to claim 13.

15. Use of the fusion protein according to any one of claims 1 to 9 or the protein dimer according to claim 10 or the pharmaceutical composition according to claim 11 in the preparation of a medicament for treating diabetes, obesity or metabolic syndrome.


SEQUENCE LISTING (23 sequences)

SEQ ID NO: 1 - protein, 30 aa, synthetic construct
  VARIANT 16: note = X is Y, A or E.
  VARIANT 25: note = X is T, I, F or H.
  VARIANT 27: note = X is E, D, T or absent.
  VARIANT 28: note = X is P, E or absent.
  VARIANT 29: note = X is K, E or absent.
  VARIANT 30: note = X is T or absent.
  VARIANT 5: note = X is H or A.
FVNQXLCGSHLVEALXLVCGERGFXYXXXX

SEQ ID NO: 2 - protein, 21 aa, synthetic construct
  VARIANT 5: note = X is Q or A.
  VARIANT 8: note = X is T, A, F or K.
  VARIANT 21: note = X is G, S or N.
  VARIANT 3: note = X is A, Lor V.
GIXEXCCXSICSLYQLENYCX

SEQ ID NO: 3 - protein, 57 aa, synthetic construct
FVNQHLCGSHLVEALYLVCGERGFFYEPKTGGVGGGGIAEQCCTSICSLYQLENYCG

SEQ ID NO: 4 - protein, 20 aa, synthetic construct
GGGGSGGGGSGGGGSGGGGS

SEQ ID NO: 5 - protein, 228 aa, synthetic construct
  VARIANT 5: note = X is S or C.
  VARIANT 8: note = X is S or C.
  VARIANT 11: note = X is S or C.
  VARIANT 49: note = X is Q or H.
  VARIANT 90: note = X is L or V.
  VARIANT 111: note = X is S or A.
  VARIANT 112: note = X is S or P.
  VARIANT 226: note = X is L or P.
  VARIANT 228: note = X is K or absent.
  VARIANT 4: note = X is S or C.
ERKXXVEXPPXPAPPVAGPSVFLFPPKPKDTLMISRTPEVTCVVVDVSXEDPEVQFNWYVDGVEVHNAKTKPREEQFNSTFRVVSVLTVXHQDWLNGKEYKCKVSNKGLPXXIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSXGX

SEQ ID NO: 6 - protein, 228 aa, synthetic construct
ESKYGPPSPPSPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - protein, 305 aa, synthetic construct
  VARIANT 16: note = X is Y, A or E.
  VARIANT 25: note = X is T, I, F or H.
  VARIANT 27: note = X is E, D, T or absent.
  VARIANT 28: note = X is P, E or absent.
  VARIANT 29: note = X is K, E or absent.
  VARIANT 30: note = X is T or absent.
  VARIANT 39: note = X is A, L or V.
  VARIANT 41: note = X is Q or A.
  VARIANT 44: note = X is T, A, F or K.
  VARIANT 57: note = X is G, S or N.
  VARIANT 62: note = X is S or T.
  VARIANT 67: note = X is S or T.
  VARIANT 72: note = X is S or T.
  VARIANT 77: note = X is S or T.
  VARIANT 81: note = X is S or C.
  VARIANT 82: note = X is S or C.
  VARIANT 85: note = X is S or C.
  VARIANT 88: note = X is S or C.
  VARIANT 126: note = X is Q or H.
  VARIANT 167: note = X is L or V.
  VARIANT 188: note = X is S or A.
  VARIANT 189: note = X is S or P.
  VARIANT 303: note = X is L or P.
  VARIANT 305: note = X is K or absent
  VARIANT 5: note = X is H or A.
FVNQXLCGSHLVEALXLVCGERGFXYXXXXGGVGGGGIXEXCCXSICSLYQLENYCXGGGGXGGGGXGGGGXGGGGXERKXXVEXPPXPAPPVAGPSVFLFPPKPKDTLMISRTPEVTCVVVDVSXEDPEVQFNWYVDGVEVHNAKTKPREEQFNSTFRVVSVLTVXHQDWLNGKEYKCKVSNKGLPXXIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSXGX

SEQ ID NO: 8 - protein, 304 aa, synthetic construct
FVNQHLCGSHLVEALYLVCGERGFFYEPKTGGVGGGGIAEQCCTSICSLYQLENYCGGGGGSGGGGSGGGGSGGGGSERKSSVESPPSPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLG

SEQ ID NO: 9 - protein, 304 aa, synthetic construct
FVNQHLCGSHLVEALYLVCGERGFFYTPKTGGVGGGGIAEQCCTSICSLYQLENYCGGGGGSGGGGSGGGGSGGGGSERKSSVESPPSPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLG

SEQ ID NO: 10 - protein, 304 aa, synthetic construct
FVNQHLCGSHLVEALYLVCGERGFFYTPKTGGVGGGGIVEACCTSICSLYQLENYCGGGGGSGGGGSGGGGSGGGGSERKSSVESPPSPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLG

SEQ ID NO: 11 - protein, 304 aa, synthetic construct
FVNQHLCGSHLVEALYLVCGERGFFYTPKTGGVGGGGIVEQCCASICSLYQLENYCGGGGGSGGGGSGGGGSGGGGSERKSSVESPPSPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLG

SEQ ID NO: 12 - protein, 304 aa, synthetic construct
FVNQALCGSHLVEALYLVCGERGFFYTPKTGGVGGGGIVEQCCTSICSLYQLENYCGGGGGSGGGGSGGGGSGGGGSERKSSVESPPSPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLG

SEQ ID NO: 13 - protein, 304 aa, synthetic construct
FVNQHLCGSHLVEALALVCGERGFFYTPKTGGVGGGGIAEQCCTSICSLYQLENYCGGGGGSGGGGSGGGGSGGGGSERKSSVESPPSPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLG

SEQ ID NO: 14 - protein, 304 aa, synthetic construct
FVNQHLCGSHLVEALYLVCGERGFTYEPKTGGVGGGGIAEQCCTSICSLYQLENYCGGGGGSGGGGSGGGGSGGGGSERKSSVESPPSPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLG

SEQ ID NO: 15 - protein, 300 aa, synthetic construct
FVNQHLCGSHLVEALYLVCGERGFFYGGVGGGGIAEQCCTSICSLYQLENYCGGGGGSGGGGSGGGGSGGGGSERKSSVESPPSPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLG

SEQ ID NO: 16 - protein, 6 aa, synthetic construct
GGVGGG

SEQ ID NO: 17 - protein, 6 aa, synthetic construct
GGGSGG

SEQ ID NO: 18 - protein, 6 aa, synthetic construct
GGGGGV

SEQ ID NO: 19 - protein, 57 aa, synthetic construct
  VARIANT 16: note = X is Y, A or E.
  VARIANT 25: note = X is T, I, F or H.
  VARIANT 27: note = X is E, D, T or absent.
  VARIANT 28: note = X is P, E or absent.
  VARIANT 29: note = X is K, E or absent.
  VARIANT 30: note = X is T or absent.
  VARIANT 39: note = X is A, L or V.
  VARIANT 41: note = X is Q or A.
  VARIANT 44: note = X is T, A, F or K.
  VARIANT 57: note = X is G, S or N.
  VARIANT 5: note = X is H or A.
FVNQXLCGSHLVEALXLVCGERGFXYXXXXGGVGGGGIXEXCCXSICSLYQLENYCX

SEQ ID NO: 20 - protein, 20 aa, synthetic construct
  VARIANT 5: note = X is S or T.
  VARIANT 10: note = X is S or T.
  VARIANT 15: note = X is S or T.
  VARIANT 20: note = X is S or T.
GGGGXGGGGXGGGGXGGGGX

SEQ ID NO: 21 - protein, 21 aa, synthetic construct
GIVEQCCTSICSLYQLENYCN

SEQ ID NO: 22 - protein, 30 aa, synthetic construct
FVNQHLCGSHLVEALYLVCGERGFFYTPKT

SEQ ID NO: 23 - protein, 304 aa, synthetic construct
FVNQHLCGSHLVEALYLVCGERGFFYEPKTGGVGGGGIVEQCCTSICSLYQLENYCNGGGGSGGGGSGGGGSGGGGSERKSSVESPPSPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLG